(19) United States
(12) Patent Application Publication
(10) Pub. No.: US 2026/0317400 A1
(43) Pub. Date: Oct. 15, 2026

(54) RECOMBINANTLY ENGINEERED, LIPASE/ESTERASE-DEFICIENT MAMMALIAN CELL LINES

(71) Applicant: Eli Lilly and Company, Indianapolis, IN (US)

(72) Inventors: Christopher Carl FRYE, Bargersville, IN (US); Troii HALL, Zionsville, IN (US); Lihua HUANG, Carmel, IN (US); Stephanie Lynn SANDEFUR, Indianapolis, IN (US)

(21) Appl. No.: 19/564,401

(22) Filed: Mar. 12, 2026

Related U.S. Application Data
(63) Continuation of application No. 17/720,993, filed on Apr. 14, 2022.
Application No. 17/720,993 is a continuation of application No. PCT/US2020/055572, filed on Oct. 14, 2020.
(60) Provisional application No. 62/915,234, filed on Oct. 15, 2019.

(51) Int. Cl. C07K 16/00; C12N 9/20

(52) U.S. Cl. CPC C07K 16/00; C12N 9/20; C07K 2317/14; C12Y 301/01013; C12Y 301/01034

(57) ABSTRACT

Mammalian cell lines with reduced expression and/or activity of lipases/esterases, and methods of producing the same are provided. Also provided are compositions comprising polysorbate and recombinant proteins produced in said mammalian cells which have improved polysorbate stability.

### SEQUENCE LISTING

The instant application contains a Sequence Listing in XML format with a file name X21790_US, created on Mar. 6, 2026, and with a file size of 23,654 bytes which is hereby incorporated by reference in its entirety.

The present invention relates to engineered mammalian cell lines, methods of producing the same, methods of producing recombinant proteins in said cell lines and compositions comprising recombinant proteins produced therein.

Mammalian cells, such as Chinese hamster ovary (CHO) cells, are widely used in the biopharmaceutical industry to produce recombinant proteins including therapeutic proteins, peptides and monoclonal antibodies (mAbs). In bioproduct manufacturing processes, concomitantly produced host cell proteins (HCPs) need to be removed or reduced in order to produce safe and effective drug, diagnostic, and/or research reagent products containing recombinant proteins. A wide variety of purification techniques have been employed to purify recombinant proteins in bioproduct manufacturing. However, HCPs can be difficult to separate from recombinant proteins produced in mammalian cells. Therefore, HCPs can present significant challenges to the production of recombinant proteins, in particular for the manufacture of therapeutic bioproducts. Methods for reducing either the expression or activity of problematic HCPs in mammalian cells used for manufacturing of bioproducts can greatly reduce the complexity of purification processes needed to manufacture recombinant proteins. Using cell lines with reduced HCPs often results in more stable, safer, and/or more effective recombinant protein-based drugs, diagnostics, and/or diagnostic research reagents.

In the production of recombinant protein products, polysorbates are often used in biomedical formulations to improve the stability of proteins during manufacture, shipment, and storage. Polysorbates can improve bioproduct stability by reducing aggregation and particle formation, specifically due to interfacial stresses, and surface adhesion of the active ingredient. However, polysorbates (which are fatty acid esters of polyoxyethylene sorbitan) in the presence of certain lipases/esterases can undergo degradation to release long-chain fatty acids. This can occur for example by ester hydrolysis. Polysorbate degradation can decrease the effectiveness of the surfactant in protecting the active pharmaceutical ingredient (API) and lead to turbidity and particle formation in the formulation over time, rendering a product incompliant, limiting its shelf life, and polysorbate degradation products may represent risks to patient safety risks. By decreasing or eliminating cellular lipases/esterases responsible for the enzymatic degradation of polysorbate detergents, the shelf life of recombinantly produced bioproduct formulations containing polysorbate detergents can be increased. Increased shelf life is important in the efficient supply of recombinant products reducing waste and enabling distribution networks.

International Patent Application Publications WO 2017/053482, WO 2016/138467, WO 2018/039499, and WO 2015/095568 describe methods of reducing the expression of problematic HCPs in mammalian cells, including various lipases/esterases. However, it is often unclear which lipases/esterases result in a specific issue related to polysorbate degradation. Accordingly, there remains a great need for engineered lipase/esterase-deficient mammalian cells that more effectively address the problem of residual mammalian cell lipase/esterase activity in recombinant protein production methods and polysorbate containing bioproduct formulations. The present invention provides, inter alia, genetically engineered host cells which enable the manufacture of bioproducts with significantly less polysorbate-degrading host cell protein contaminants, resulting in significantly improved stability in polysorbate containing bioproduct formulations.

In one aspect, a mammalian cell is provided which has reduced expression and/or activity of at least one endogenous palmitoyl-protein thioesterase (PPT) and at least one HCP selected from the group consisting of a lysosomal acid lipase (LAL), a lipoprotein lipase (LPL), a phospholipase A2, and a phospholipase D.

In another aspect a process for reducing polysorbate degradation in a protein formulation is provided which comprises the steps of:
(a) modifying a host cell to reduce or eliminate the expression of palmitoyl-protein thioesterase 1 (PPT1) protein;
(b) modifying the host cell to reduce or eliminate the expression of lysosomal acid lipase (LAL), lipoprotein lipase (LPL), phospholipase D3 (PLD3), and/or phospholipase A2 (LPLA2);
(c) transfecting the cell with a polynucleotide encoding a bioproduct;
(d) extracting a protein fraction comprising the protein of interest from the host cell;
(e) contacting the protein fraction with a chromatography media which is protein A affinity (PA) chromatography or another affinity chromatography method, cation exchange (CEX) chromatography, anion exchange (AEX) chromatography or hydrophobic interaction chromatography (HIC); and
(f) collecting the protein of interest from the media;
(g) combining the bioproduct with a fatty acid ester; and
(h) optionally, adding a buffer; and
(i) optionally, adding one or more pharmaceutically acceptable carriers, diluents, or excipients.

In another aspect, a process for reducing aggregation or particle formation in a protein formulation is provided which comprises the steps of:
(a) modifying a host cell to reduce or eliminate the expression of palmitoyl-protein thioesterase 1 (PPT1) protein;
(b) modifying the host cell to reduce or eliminate the expression of lysosomal acid lipase (LAL), lipoprotein lipase (LPL), phospholipase D3 (PLD3), and/or phospholipase A2 (LPLA2);
(c) transfecting the cell with a polynucleotide encoding a bioproduct of interest;
(d) extracting a protein fraction comprising the protein of interest from the host cell;
(e) contacting the protein fraction with a chromatography media which is protein A affinity (PA) chromatography or another affinity chromatography method, cation exchange (CEX) chromatography, anion exchange (AEX) chromatography or hydrophobic interaction chromatography (HIC); and
(f) collecting the protein of interest from the media; and
(g) combining the protein of interest with a fatty acid ester; and
(h) optionally, adding a buffer; and
(i) optionally, adding one or more pharmaceutically acceptable carriers, diluents, or excipients.

In another aspect, a process for producing a stable formulated bioproduct is provided which comprises the steps of:
(a) modifying a host cell to reduce or eliminate the expression of palmitoyl-protein thioesterase 1 (PPT1) protein;
(b) modifying the host cell to reduce or eliminate the expression of lysosomal acid lipase (LAL), lipoprotein lipase (LPL), phospholipase D3 (PLD3), and/or phospholipase A2 (LPLA2);
(c) transfecting the cell with a polynucleotide encoding a bioproduct;
(d) extracting a protein fraction comprising the bioproduct from the host cell;
(e) contacting the protein fraction with a chromatography media which is protein A affinity (PA) chromatography or another affinity chromatography method, cation exchange (CEX) chromatography, anion exchange (AEX) chromatography or hydrophobic interaction chromatography (HIC);
(f) collecting the bioproduct from the media;
(g) combining the bioproduct with a fatty acid ester;
(h) optionally, adding a buffer; and
(i) optionally, adding one or more pharmaceutically acceptable carriers, diluents, or excipients.

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments including, but not limited to Fc fragments, or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The term “fatty acid hydrolase” or “FAH” as used herein is intended to refer to any hydrolytic enzyme that cleaves at a carbonyl group creating a carboxylic acid product in which the carboxylic acid comprises an R-group that is lipophilic or otherwise hydrophobic. In cases, the carboxylic acid product is a fatty acid.

The term “polysorbate” refers to nonionic surfactants which are fatty acid esters of polyethoxylated sorbitan. Examples of polysorbates used in biomedical formulations include, but are not limited to, Polysorbate 80 (PS80), Polysorbate 20 (PS20), Polysorbate 40 (PS40), Polysorbate 60 (PS60), Polysorbate 65 (PS65), or a combination thereof. The concentration of polysorbate in the pharmaceutical compositions of the present invention, may be at about 0.01% to about 1%, preferably, about 0.01% to about 0.10%, more preferably, about 0.01% to about 0.05%, even more preferably, about 0.02% to about 0.05% by weight in the composition of the present invention.

The term “lipase/esterase” as used herein is intended to mean the group of mammalian cell enzymes consisting of both “esterases” and “lipases”. “Esterases” are a subgenus of fatty acid hydrolases that cleave fatty acid esters into fatty acids and alcohols. “Lipases” are a subgenus of esterases that cleave lipids (fats, waxes, sterols, glycerides and phospholipids). “Phospholipases” are a subgenus of lipases that cleave phospholipids.

Palmitoyl-protein thioesterase 1 (PPT1) is a member of the palmitoyl protein thioesterase family and is a lysosomal enzyme involved in the catabolism of lipid-modified proteins during lysosomal degradation and which cleaves the thioester formed from the fatty acid palmitate from cysteine residues in proteins. In an embodiment, Chinese hamster PPT1 comprises an amino acid sequence of SEQ ID NO:1. In an embodiment, PPT1 is modified by ZFN at a binding/cutting region nucleic acid sequence of SEQ ID NO:8. Lysosomal acid lipase (LAL), also known as lysosomal lipase, lipase A, lysosomal acid and cholesterol esterase is an intracellular lipase that functions in lysosomes. LAL catalyzes cholesteryl ester bond cleavage. In an embodiment, Chinese hamster LAL comprises an amino acid sequence of SEQ ID NO:2. In an embodiment, LAL is modified by ZFN at a binding/cutting region nucleic acid sequence of SEQ ID NO:7.

Lipoprotein lipase isoform X2 (herein referred to as LPL) is a glycosylated homodimer secreted by parenchymal cells and associated with endothelial cells of the capillary lumen. In an embodiment, Chinese hamster LPL comprises an amino acid sequence of SEQ ID NO:3. In an embodiment, LPL is modified by ZFN at a binding/cutting region nucleic acid sequence of SEQ ID NO:6.

Group XV lysosomal phospholipase A2 isoform X1 (herein referred to as LPLA2) is a member of a family of key lipid-metabolizing enzymes and cleaves fatty acids from the sn-2 position of membrane phospholipids. In an embodiment, Chinese hamster LPLA2 comprises an amino acid sequence of SEQ ID NO:4. In an embodiment, LPLA2 is modified by ZFN at a binding/cutting region nucleic acid sequence of SEQ ID NO:5

PLD3 is a member of the phospholipase D (PLD) lipid-signaling enzyme superfamily. PLD family members are known to hydrolyze phosphatidylcholine to give phosphatidic acid and choline. PLD3 is a N-glycosylated type II transmembrane protein which retains HKD motifs shown to confer phosphodiester hydrolytic activity in other PLD family members (e.g. PLD1 and PLD2). In an embodiment, Chinese hamster PLD3 comprises an amino acid sequence of SEQ ID NO:9. In an embodiment, PLD3 is modified by ZFN at a binding/cutting region nucleic acid sequence of SEQ ID NO:10.

The terms “mammalian cells” and “host cells” are used interchangeably herein and to refer to mammalian cells which are commonly used in the production of bioproducts using recombinant DNA technology. For example, chinese hamster ovary (CHO) cells, human embryonic kidney 293 (HEK 293), and mouse myeloma cells, including NS0 and Sp2/0 cells, are commonly used mammalian cells for protein expression. Preferably, the mammalian cell is CHO, including, but not limited to, CHO-K1, CHO pro-3, DUKX-X11, DG44, CHOK1SV or CHOK1SV GS-KO. The parental cell line may be also modified by the insertion, knock-out or knock-down of genes that affect the critical quality attributes or other post-translational modifications of a recombinant bioproduct polypeptide, or the expression of the gene encoding the recombinant bioproduct. In embodiments, the host cell is a Chinese hamster ovary (CHO) cell. In one embodiment, the host cell is a CHO-K1 cell, a CHOK1SV cell, a DG44 CHO cell, a DUXB11 CHO cell, a CHO-S, a CHO GS knock-out cell (glutamine synthetase), a CHOK1SV FUT8 knock-out cell, a CHOZN, or a CHO-derived cell. The CHO GS knock-out cell (e.g., GSKO cell) is, for example, a CHO-K1SV GS knockout cell (Lonza Biologics, Inc.). The CHO FUT8 knockout cell is, for example, the Potelligent® CHOK1SV FUT8 knock-out (Lonza Biologics, Inc.). In embodiments, the host cell is a HeLa, MDCK, Sf9, Sf21, Tn5, HT1080, NB324K, FLYRD18, HEK293, HEK293T, HT1080, H9, HepG2, MCF7, Jurkat, NIH3T3, PC12, PER.C6, BHK (baby hamster kidney), VERO, SP2/0, NS0, YB2/0, Y0, EB66, C127, L cell, COS (e.g., COSI and COS7), QC1-3, CHOK1, CHOK1SV, Potelligent™ (CHOKlSV FUT8-KO), CHO GS knockout, Xceed™ (CHOKlSV GS-KO), CHOS, CHO DG44, CHO DXB11, or CHOZN cell, or any cells derived therefrom.

The term “parental cell line” herein refers to a non-transgenic protein product expressing mammalian cell commonly used for engineering protein expression. In some embodiments of the present invention, the parental cell line is a CHO, HEK293, or a NS0 cell line. Preferably, the parental cell line is a CHO cell line, including, but not limited to, a GS-CHO (CHOK1SV or CHOK1SV GS-KO) cell line.

The term “product expressing cell line” refers to a “parental cell line” into which one or more genes encoding at least one bioproduct has been inserted and which is capable of expressing such protein or proteins. Preferably, the “product expressing cell line” expresses an antibody, or an antigen-binding fragment thereof.

The term “indel” refers to insertion or deletion of nucleic acid bases in the genome of a cell.

The term “bioproduct” as used herein refers to recombinant protein-based products of interest derived from genetically engineered mammalian cells using recombinant DNA technologies. For example, bioproducts may include antibodies, antigen-binding fragments thereof, vaccines, growth factors, cytokines, hormones, peptides, enzymes, fusion proteins. Preferably, bioproducts are useful therapeutically, diagnostically, industrially, and/or for research applications.

The term “inactivated gene” refers to a gene which has been altered in such a way that it 1) does not express detectable levels of the protein originally encoded by the unaltered wild-type gene; and/or 2) the protein encoded by the altered gene is phenotypically non-functional as compared to the protein originally encoded by the un-altered wild-type gene.

The term “disrupted gene” refers to a gene which has been altered in such a way that 1) the expression of the protein which the un-altered wild-type gene originally encoded is reduced, and/or 2) the activity of the protein encoded by the altered gene is reduced as compared to the activity of the protein encoded by the unaltered wild-type gene.

The terms “protein” and “polypeptide” are used interchangeably herein to refer to a polymer of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, proteins containing one or more analogs of an amino acid (including, for example, unnatural amino acids, etc.), as well as other modifications known in the art. Examples of proteins include, but are not limited to, antibodies, peptides, enzymes, receptors, hormones, regulatory factors, antigens, binding agents, cytokines, Fc fusion proteins (e.g. an Fc domain of an IgG which is genetically linked to a peptide/protein of interest), immunoadhesin molecules, etc.

In one aspect of the present invention, a mammalian cell is provided which has reduced expression and/or activity of at least one endogenous palmitoyl-protein thioesterase (PPT) and at least one HCP selected from the group consisting of a lysosomal acid lipase (LAL), a lipoprotein lipase (LPL), a phospholipase A2, and a phospholipase D. In another aspect of the invention, the mammalian cell is further modified to express at least one bioproduct. The bioproduct may be, for example, 1) a polypeptide, 2) an antibody, or a fragment thereof, including, but not limited to, an antigen-binding fragment thereof, or 3) a protein-protein fusion, including, but not limited to, an Fc-fusion protein.

In one aspect of the present invention, a mammalian cell is provided which has reduced expression and/or activity of endogenous palmitoyl-protein thioesterase 1 (PPT1) and at least one HCP selected from the group consisting of lysosomal acid lipase (LAL), lipoprotein lipase (LPL), phospholipase A2 (LPLA2), and phospholipase D3 (PLD3).

In one aspect to the present invention, a mammalian cell is provided which has a modification in a coding sequence of a polynucleotide encoding the lysosomal acid lipase (LAL) protein, the lipoprotein lipase (LPL) protein, the phospholipase A2 (LPLA2) protein, and the palmitoyl-protein thioesterase 1 (PPT1) protein, wherein the modification decreases the expression level of the LAL protein, the LPL protein, the LPLA2 protein, and the PPT1 protein in a cell having the modification relative to the expression level of a cell without any of said modifications.

In another aspect of the invention, the mammalian cell is further modified to express at least one bioproduct. The bioproduct may be, for example, 1) a polypeptide, 2) an antibody, or a fragment thereof, including, but not limited to, an antigen-binding fragment thereof, or 3) an Fc-fusion protein.

In another aspect of the invention, a mammalian cell is provided in which the cell's genes encoding endogenous PPT and at least one other polysorbate degrading HCP selected from the group consisting of LAL, LPL, LPLA2, and PLD3 has been modified such that the expression and/or activity of the endogenous PPT1 and the other selected HCPs is reduced. Preferably, the activity and/or expression of the endogenous PPT1 and at least one HCP selected from the group consisting of LAL, LPL, LPLA2, and PLD3 has been substantially reduced or eliminated entirely. In another aspect, a method is provided for producing a mammalian cell in which the gene encoding endogenous PPT1 and at least one HCP selected from the group consisting of LAL, LPL, LPLA2 and PLD3 have been modified such that the expression and/or activity of those HCPs is reduced. Preferably, the activity and/or expression of the endogenous PPT1 and at least one HCP selected from the group consisting of LAL, LPL, LPLA2, and PLD3 has been substantially reduced or eliminated entirely. In another aspect of the invention is provided a method of producing a recombinant protein in an embodiment of a mammalian cell as described herein. Material produced from the mammalian cell embodiments described herein shows no or significantly reduced hydrolytic polysorbate degradation, and essentially no relevant lipase activity can be measured (such as with a lipolytic activity assay).

In some embodiments, bioproducts produced from mammalian cells of the present invention provides protein A-binding fractions having substantially reduced polysorbate degradation activity relative to the polysorbate degradation activity of the same bioproduct produced in an essentially similar cell without any of the modifications. In some embodiments, the reduction in degradation of intact polysorbate arising from a bioproduct produced in a product expressing cell line of the invention relative to the degradation of intact polysorbate arising from the same bioproduct produced in the corresponding unmodified product expressing cell line is greater than about 20%, greater than 25%, greater than about 30%, greater than about 35%, greater than about 40%, greater than about 45%, greater than about 50%, greater than about 55%, greater than about 60%, greater than about 65%, greater than about 70%, greater than about 75%, or greater than about 80%. In some embodiments, the reduction in degradation of intact polysorbate arising from a bioproduct produced in a product expressing cell line of the invention relative to the degradation of intact polysorbate arising from the same bioproduct produced in the corresponding unmodified product expressing cell line is greater than 20%, greater than 25%, greater than 30%, greater than 35%, greater than 40%, greater than 45%, greater than 50%, greater than 55%, greater than 60%, greater than 65%, greater than 70%, greater than 75%, or greater than 80%.

In some embodiments, the reduction in degradation of intact polysorbate arising from a bioproduct produced in a product expressing cell line of the invention relative to the degradation of intact polysorbate arising from the same bioproduct produced in the corresponding unmodified product expressing cell line is between about 20% to about 80%, between about 30% to about 75%, between about 35% to about 70%, between about 40% to about 65%, or between about 45% and about 60%.

In some embodiments, the reduction in degradation of intact polysorbate arising from a bioproduct produced in a product expressing cell line of the invention relative to the degradation of intact polysorbate arising from the same bioproduct produced in the corresponding unmodified product expressing cell line is between 20%-80%, between 30%-75%, between 35%-70%, between 40%-65%, and between 45%-60%.

In one aspect of the invention, gene-editing methods are employed to target the gene encoding endogenous PPT1 and the gene(s) encoding at least one HCP selected from the group consisting of LAL, LPL, LPLA2, and PLD3 in order to edit, disrupt, and/or inactivate them, e.g., due to modification, insertion, or deletion of the genomic loci. In some embodiments, one or both alleles of the endogenous host cell protein, PPT1, and at least one HCP selected from the group consisting of LAL, LPL, LPLA2, and PLD3 are knocked out from the genome of the engineered host cells described herein (e.g., CHO cells). For example, gene-editing methods include, but are not limited to, use of zinc-finger nuclease (ZFN), clustered, regularly interspaced, short palindromic repeats (CRISPR), transcription activator-like effector nuclease (TALEN), and meganuclease systems.

In one aspect of the invention, a recombinantly engineered mammalian cell is provided which comprises modifications in polynucleotide sequences encoding the LAL protein, the LPL protein, the LPLA2 protein, and the endogenous PPT1 protein. In another aspect of the invention, the modification decreases the expression level of the LAL protein, the LPL protein, the LPLA2 protein, and the PPT1 protein as compared to the expression level of a cell lacking the modifications, e.g., the wild type mammalian cell.

In some embodiments, the target HCP gene is edited, disrupted, and/or inactivated by a gene deletion. As used herein, “gene deletion” refers to removal of at least a portion of a DNA sequence from, or in proximity to, a gene. In some embodiments, the sequence subjected to gene deletion comprises an exonic sequence of a gene. In some embodiments, the sequence subjected to gene deletion comprises a promoter sequence of a gene. In some embodiments, the sequence subjected to gene deletion comprises a flanking sequence of a gene. In some embodiments, the sequence subjected to gene deletion comprises a sequence encoding the signal peptide of the targeted HCP. In some embodiments, a portion of a target HCP gene sequence is removed from the target HCP gene, or from a region in relatively close proximity to the target HCP gene. In some embodiments, the complete target HCP gene sequence is removed from a chromosome. In some embodiments, the mammalian cell comprises a gene deletion in proximity to the target HCP gene. In some embodiments, the target HCP gene is edited, disrupted, and/or inactivated by a gene deletion, wherein deletion of at least one nucleotide or nucleotide base pair in a gene sequence results in a non-functional gene product. In some embodiments, the target HCP gene is edited, disrupted, and/or inactivated by a gene deletion, wherein deletion of at least one nucleotide of the gene sequence results in a gene product that no longer has the original gene product function or activity, or is dysfunctional.

In some embodiments, the target HCP gene is edited, disrupted, and/or inactivated by a gene addition or substitution. As used herein, “gene addition” or “gene substitution” refers to an alteration of a target HCP gene sequence, including insertion or substitution of one or more nucleotides or nucleotide base pairs. In some embodiments, the intronic sequence of the target HCP gene is altered. In some embodiments, the exonic sequence of the target HCP gene is altered. In some embodiments, the promoter sequence of the target HCP gene is altered. In some embodiments, the flanking sequence of the target HCP gene is altered. In some embodiments, the sequence encoding the target HCP's signal peptide is altered. In some embodiments, one nucleotide or nucleotide base pair is added to a target HCP gene sequence. In some embodiments, at least one consecutive nucleotide or nucleotide base pair is added to a target HCP gene sequence. In some embodiments, the target HCP gene is inactivated by a gene addition or substitution, wherein addition or substitution of at least one nucleotide or nucleotide base pair into the target HCP gene sequence results in a non-functional gene product. In some embodiments, the target HCP gene is inactivated by a gene inactivation, wherein incorporation or substitution of at least one nucleotide to the target HCP gene sequence results in a gene product that no longer has the original gene product function or activity, or is dysfunctional.

Generally, a CRISPR system comprises a caspase protein, such as Cas9, and an RNA sequence comprising a nucleotide sequence, referred to as a guide sequence, that is complementary to a sequence of interest. The caspase and RNA sequence form a complex that identify a DNA sequence of a mammalian cell, and subsequently the nuclease activity of the caspase allows for cleavage of the DNA strand. Caspase isotypes have single-stranded DNA or double-stranded DNA nuclease activity. Design of guide RNA sequences and number of guide RNA sequences used in a CRISPR system allow for removal of a specific stretch of a gene and/or addition of a DNA sequence.

In some embodiments, the methods of the present invention comprise editing, disrupting, and/or inactivating the gene encoding endogenous PPT1 and the gene(s) encoding at least one HCP selected from the group consisting of LAL, LPL, LPLA2, and PLD3 using at least one genome editing system selected from the group consisting of a CRISPR, TALEN, ZFN, and a meganuclease system.

Generally, a TALEN system comprises one or more restriction nucleases and two or more protein complexes that allow for recognition of a DNA sequence and subsequent double-stranded DNA cleavage. A protein complex of the TALEN system comprises a number of transcription activator-like effectors (TALEs), each recognizing a specific nucleotide, and a domain of a restriction nuclease. Generally, a TALEN system is designed so that two protein complexes, each comprising TALEs and a domain of a restriction nuclease, will individually bind to DNA sequences in a manner to allow for the two domains (one from each protein complex) of a restriction nuclease to form an active nuclease and cleave a specific DNA sequence. Design of number of protein complexes and sequences to be cleaved in a TALEN system allows for removal of a specific stretch of a gene and/or addition of a DNA sequence.

In some embodiments, the methods of the present invention comprise editing, disrupting, and/or inactivating the gene encoding endogenous PPT1 and the gene(s) encoding at least one HCP selected from the group consisting of LAL, LPL, LPLA2, and PLD3 using a TALEN system.

In some embodiments, the method of producing a mammalian cell, wherein the mammalian cell has a reduced level of endogenous PPT1 and a reduced level of at least one HCP selected from the group consisting of LAL, LPL, LPLA2, and PLD3, comprises editing, disrupting, and/or inactivating endogenous PPT1 and at least one of the other target HCP genes (i.e., LAL, LPL, LPLA2, and PLD3), using a TALEN system.

Generally, a ZFN system comprises one or more restriction nucleases and two or more protein complexes that allow for recognition of a DNA sequence and subsequent double-stranded DNA cleavage. A protein complex of the ZFN system comprises a number of zinc fingers, each recognizing a specific nucleotide codon, and a domain of a restriction nuclease. Generally, a ZFN system is designed so that two protein complexes, each comprising zinc fingers and a domain of a restriction nuclease, will individually bind to DNA sequences in a manner to allow for the two domains (one from each protein complex) of a restriction nuclease to form an active nuclease and cleave a specific DNA sequence. Design of number of protein complexes and sequences to be cleaved in a ZFN system allows for removal of a specific stretch of a gene and/or addition of a DNA sequence.

In some embodiments, the methods of the present invention comprise editing, disrupting, and/or inactivating the gene encoding endogenous PPT1 and the gene(s) encoding at least one HCP selected from the group consisting of LAL, LPL, LPLA2, and PLD3 using a ZFN system.

In some embodiments, the method of producing a mammalian cell, wherein the mammalian cell has a reduced level of endogenous PPT1 and a reduced level of at least one HCP selected from the group consisting of LAL, LPL, LPLA2, and PLD3, comprises editing, disrupting, and/or inactivating endogenous PPT1 and at least one of the other target HCP genes (i.e., LAL, LPL, LPLA2, and PLD3), using a ZFN system.

Generally, a meganuclease system comprises one or more meganucleases that allow for recognition of a DNA sequence and subsequent double-stranded DNA cleavage.

In some embodiments, the methods of the present invention comprise editing, disrupting, and/or inactivating the gene encoding endogenous PPT1 and the gene(s) encoding at least one HCP selected from the group consisting of LAL, LPL, LPLA2, and PLD3 using a meganuclease system.

In some embodiments, the method of producing a mammalian cell, wherein the mammalian cell has a reduced level of endogenous PPT1 and a reduced level of at least one HCP selected from the group consisting of LAL, LPL, LPLA2, and PLD3, comprises editing, disrupting, and/or inactivating endogenous PPT1 and at least one of the other target HCP genes (i.e., LAL, LPL, LPLA2, and PLD3), using a meganuclease system.

The engineered host cells described herein (e.g., CHO cells) can include additional genomic modifications to alter the glycosylation patterns of the antibodies produced in those cells. Altered glycosylation patterns, such as reduced fucosylation, have been demonstrated to increase the antibody-dependent cellular cytotoxicity (ADCC) activities of antibodies. For example, host cells with knockout of both alleles of FUT8 (fucosyltransferase 8, or a-1,6-fucosyltransferase) can produce antibodies with enhanced ADCC activity (see U.S. Pat. No. 6,946,292). In some embodiments, the engineered host cells described herein (e.g., CHO cells) include gene modifications that reduce fucosylation of antibodies. In some embodiments, the engineered host cells described herein (e.g., CHO cells) comprise an edited, disrupted, and/or inactivated FUT8 gene, e.g., due to modification, insertion, or deletion of the FUT8 genomic locus. In some embodiments, one or both alleles of FUT8 are knocked out from the genome of the engineered host cells described herein (e.g., CHO cells). Antibodies produced in such FUT8 knockout host cells may have increased ADCC activity. Other enzymes responsible for glycosylation include GDP-mannose 4,6-dehydratase, GDP-keto-6-deoxymannose 3,5-epimerase 4,6-reductase, GDP-beta-L-fucose pyrophosphorylase, N-acetylglucosaminyltransferase III, and fucokinase. In some embodiments, the engineered host cells described herein (e.g., CHO cells) may comprise an inactivated gene encoding one or more of these enzymes. In an embodiment, Chinese hamster FUT8 comprises an amino acid sequence of SEQ ID NO:11.

The engineered host cells described herein (e.g., CHO cells) can also include additional genomic modifications which affect the stability of recombinant proteins which they express. For example, cathepsin D (CatD) has been identified as a CHO HCP involved in degradation of Fc-fusion recombinant proteins (see Robert, F.; et al. “Degradation of an Fc-Fusion Recombinant Protein by Host Cell Proteases: Identification of a CHO Cathepsin D Protease.” Biotechnology and Bioengineering 2009, 104(6), 1132-1141). In some embodiments, the engineered host cells described herein (e.g., CHO cells) comprise an edited, disrupted, and/or inactivated CatD gene, e.g., due to modification, insertion, or deletion of the CatD genomic locus. In some embodiments, one or both alleles of CatD are knocked out from the genome of the engineered host cells described herein (e.g., CHO cells). Recombinant proteins produced in such knockout host cells may experience less degradation during production. In an embodiment, Chinese hamster CatD comprises an amino acid sequence of SEQ ID NO: 12. In an embodiment, CatD is modified by ZFN at a binding/cutting region nucleic acid sequence of SEO ID NO:13.

The engineered host cells described herein (e.g., CHO cells) can also include additional genomic modifications which affect the heterogeneity of the recombinant proteins which they express. For example, carboxypeptidase D (CpD) is capable of cleaving the C-terminal lysine from IgG1, IgG2, and IgG4 monoclonal antibody isotypes (see International Patent Application Publication WO 2017/053482). This can lead to charge variants, which can add complexity to manufacturing control strategies. In some embodiments, the engineered host cells described herein (e.g., CHO cells) comprise an edited, disrupted, and/or inactivated CpD gene, e.g., due to modification, insertion, or deletion of the CpD genomic locus. In some embodiments, one or both alleles of CpD are knocked out from the genome of the engineered host cells described herein (e.g., CHO cells). Recombinant proteins produced in such knockout host cells may have decreased charge variant heterogeneity. In an embodiment, Chinese hamster CpD comprises an amino acid sequence of SEQ ID NO:14. In an embodiment, CpD is modified by ZFN at a binding/cutting region nucleic acid sequence of SEQ ID NO:15.

The engineered host cells described herein (e.g., CHO cells) can also include additional genomic modifications which affect the downstream processes used in manufacturing recombinant proteins. For example phospholipase B-like 2 (PLBL2) and peroxiredoxin-1 (PRDX1) are HCPs which have been identified as contaminants in recombinant proteins produced in CHO cells after protein capture chromatography (see WO 2016/138467 and Doneanu, C.; et al. “Analysis of host-cell proteins in biotheraputic proteins by comprehensive online two-dimensional liquid chromatography/mass spectrometry.” mAbs 2012, 4(1), 24-44). In some embodiments, the engineered host cells described herein (e.g., CHO cells) comprise an edited, disrupted, and/or inactivated gene or genes encoding one or both of the proteins in the group consisting of PLBL2 and PRDX1, e.g., due to modification, insertion, or deletion of the genomic locus or loci. In some embodiments, one or both alleles of a gene or genes encoding one or both of the proteins in the group consisting of PLBL2 and PRDX1 are knocked out from the genome of the engineered host cells described herein (e.g., CHO cells). Recombinant proteins produced in such knockout host cells may have decreased HCP contamination relative to wild type and may require fewer downstream purification steps. In an embodiment, Chinese hamster PLBL2 comprises an amino acid sequence of SEQ ID NO:16. In an embodiment, PLBL2 is modified by ZFN at a binding/cutting region nucleic acid sequence of SEQ ID NO: 17. In an embodiment, Chinese hamster PRDX1 comprises an amino acid sequence of SEQ ID NO:18. In an embodiment, PRDX1 is modified by ZFN at a binding/cutting region nucleic acid sequence of SEQ ID NO: 19.

In some embodiments, the mammalian cells of the present invention (e.g., CHO cells) encode a recombinant protein which is tanezumab (see e.g., WO 2004/058184).

In some embodiments, the mammalian cells of the present invention (e.g., CHO cells) encode a recombinant protein which is lebrikizumab (see e.g., WO 2005/062967).

In some embodiments, the mammalian cells of the present invention (e.g., CHO cells) encode a recombinant protein which is mirikizumab (see e.g., WO 2014/137962).

In some embodiments, the mammalian cells of the present invention (e.g., CHO cells) encode a recombinant protein which is solanezumab (see e.g., WO 2001/62801).

In some embodiments, the mammalian cells of the present invention (e.g., CHO cells) encode a recombinant protein which is donanemab (see e.g., WO 2012/021469).

In some embodiments, the mammalian cells of the present invention (e.g., CHO cells) encode a recombinant protein which is zagotenemab (see e.g., WO 2016/137811).

In some embodiments, the mammalian cells of the present invention (e.g., CHO cells) encode a recombinant protein which is ramucirumab (see e.g., WO 2003/075840).

In some embodiments, the mammalian cells of the present invention (e.g., CHO cells) encode a recombinant protein which is galcanezumab (see e.g., WO 2011/156324).

In some embodiments, the mammalian cells of the present invention (e.g., CHO cells) encode a recombinant protein which is ixekizumab (see e.g., WO 2007/070750).

In some embodiments, the mammalian cells of the present invention (e.g., CHO cells) encode a recombinant protein which is dulaglutide (see e.g., WO 2005/000892).

In some embodiments, the mammalian cells of the present invention (e.g., CHO cells) encode a recombinant protein which is necitumumab (see e.g., WO 2005/090407).

In some embodiments, the mammalian cells of the present invention (e.g., CHO cells) encode a recombinant protein which is olaratumab (see e.g., WO 2006/138729).

In some embodiments, the mammalian cells of the present invention (e.g., CHO cells) encode a recombinant protein which is cetuximab.

In some embodiments, the mammalian cells of the present invention (e.g., CHO cells) encode a recombinant protein which is an angiopoietin 2 mAb (see e.g., WO 2015/179166).

In some embodiments, the mammalian cells of the present invention (e.g., CHO cells) encode a recombinant protein which is an insulin-Fc fusion protein (see e.g., WO 2016/178905).

In some embodiments, the mammalian cells of the present invention (e.g., CHO cells) encode a recombinant protein which is a CD200R agonist antibody (see e.g., WO 2020/055943).

In some embodiments, the mammalian cells of the present invention (e.g., CHO cells) encode a recombinant protein which is an epiregulin/transforming growth factor alpha (epiregulin/TGFα) mAb (see e.g., WO 2012/138510).

In some embodiments, the mammalian cells of the present invention (e.g., CHO cells) encode a recombinant protein which is an angiopoietin-like 3/8 (ANGPTL 3/8) antibody (see e.g., WO 2020/131264).

In some embodiments, the mammalian cells of the present invention (e.g., CHO cells) encode a recombinant protein which is a B- and T-lymphocyte attenuator (BTLA) antibody agonist (see e.g., WO 2018/213113).

In some embodiments, the mammalian cells of the present invention (e.g., CHO cells) encode a recombinant protein which is a CXC chemokine receptor 1/2 (CXCR1/2) ligands antibody (see e.g., WO 2014/149733).

In some embodiments, the mammalian cells of the present invention (e.g., CHO cells) encode a recombinant protein which is a growth/differentiation factor 15 (GDF15) agonist (see e.g., WO 2019/195091).

In some embodiments, the mammalian cells of the present invention (e.g., CHO cells) encode a recombinant protein which is an interleukin 33 (IL-33) antibody (see e.g., WO 2018/081075).

In some embodiments, the mammalian cells of the present invention (e.g., CHO cells) encode a recombinant protein which is a pituitary adenylate cyclase-activating polypeptide-38 (PACAP38) antibody (see e.g., WO 2019/067293).

In some embodiments, the mammalian cells of the present invention (e.g., CHO cells) encode a recombinant protein which is a programmed cell death-1 (PD-1) antibody agonist (see e.g., WO 2017/025016).

In some embodiments, the mammalian cells of the present invention (e.g., CHO cells) encode a recombinant protein which is a pyroglutamate-Abeta (pGlu-Abeta, also called N3pG Abeta) mAb (see e.g., WO 2012/021469).

In some embodiments, the mammalian cells of the present invention (e.g., CHO cells) encode a recombinant protein which is a tumor necrosis factor alpha/interleukin 23 (TNFα/IL-23) bispecific antibody (see e.g., WO 2019/027780).

In some embodiments, the mammalian cells of the present invention (e.g., CHO cells) encode a recombinant protein which is an anti-alpha-synuclein antibody (see e.g., WO 2020/123330).

In some embodiments, the mammalian cells of the present invention (e.g., CHO cells) encode a recombinant protein which is a cluster of differentiation 226 (CD226) agonist antibody (see e.g., WO 2020/023312).

In some embodiments, the mammalian cells of the present invention (e.g., CHO cells) encode a recombinant protein which is a monocarboxylate transporter 1 (MCT1) antibody (see e.g., WO 2019/136300).

In some embodiments, the mammalian cells of the present invention (e.g., CHO cells) encode a recombinant protein which is a severe acute respiratory syndrome coronavirus 2 (SARS-CoV-2) neutralizing antibody.

In some embodiments, the mammalian cells of the present invention (e.g., CHO cells) encode a recombinant protein which is an anti-Fc gamma receptor IIB (FcgRIIB or FcγRIIB) antibody.

In some embodiments, the mammalian cells of the present invention (e.g., CHO cells) encode a recombinant protein which is an anti-interleukin 34 (IL-34) antibody.

In some embodiments, the mammalian cells of the present invention (e.g., CHO cells) encode a recombinant protein which is an anti-cluster of differentiation 19 (CD19) antibody.

In some embodiments, the mammalian cells of the present invention (e.g., CHO cells) encode a recombinant protein which is a triggering receptor expressed on myeloid cells 2 (TREM2) antibody.

In some embodiments, the mammalian cells of the present invention (e.g., CHO cells) encode a recombinant protein which is a relaxin analog.

In some embodiments, the mammalian cells of the present invention (e.g., CHO cells) encode a recombinant protein which is selected from the group consisting of tanezumab, lebrikizumab, mirikizumab, solanezumab, donanemab, zagotenemab, ramucirumab, galcanezumab, ixekizumab, dulaglutide, necitumumab, olaratumab, cetuximab, an angiopoietin 2 mAb, an insulin-Fc fusion protein, CD200R agonist antibody, epiregulin/TGFα mAb, ANGPTL 3/8 antibody, a BTLA antibody agonist, a CXCR1/2 ligands antibody, a GDF15 agonist, an IL-33 antibody, a PACAP38 antibody, a PD-1 agonist antibody, pGlu-Abeta, also called N3pG Abeta mAb, a TNFα/IL-23 bispecfic antibod, an anti-alpha-synuclein antibody, CD226 agonist antibody, MCT1 antibody, a SARS-CoV-2 neutralizing antibody, an FcgRIIB antibody, an IL-34 antibody, a CD19 antibody, a TREM2 antibody, and a relaxin analog.

An embodiment of the invention also provides a pharmaceutical composition comprising a polysorbate and a bioproduct selected from the group consisting of tanezumab, lebrikizumab, mirikizumab, solanezumab, donanemab, zagotenemab, ramucirumab, galcanezumab, ixekizumab, dulaglutide, necitumumab, olaratumab, cetuximab, an angiopoietin 2 mAb, an insulin-Fc fusion protein, CD200R agonist antibody, epiregulin/TGFα mAb, ANGPTL 3/8 antibody, a BTLA antibody agonist, a CXCR1/2 ligands antibody, a GDF15 agonist, an IL-33 antibody, a PACAP38 antibody, a PD-1 agonist antibody, pGlu-Abeta, also called N3pG Abeta mAb, a TNFα/IL-23 bispecfic antibodies, an anti-alpha-synuclein antibody, CD226 agonist antibody, MCT1 antibody, a SARS-CoV-2 neutralizing antibody, an FcgRIIB antibody, an IL-34 antibody, a CD19 antibody, a TREM2 antibody, and a relaxin analog, wherein the bioproduct was produced by the recombinant mammalian cells of the present invention. In various embodiments the polysorbate is Polysorbate 80 (PS80), Polysorbate 20 (PS20), Polysorbate 40 (PS40), Polysorbate 60 (PS60), Polysorbate 65 (PS65), or a combination thereof. The concentration of polysorbate in the pharmaceutical compositions of the present invention, may be at about 0.010% to about 1%, preferably, about 0.01% to about 0.10%, more preferably, about 0.01% to about 0.05%, even more preferably, about 0.02% to about 0.05% by weight/volume (w/v) in the composition of the present invention. In other embodiments, the pharmaceutical compositions of the present invention further comprise one or more pharmaceutically acceptable carriers, diluents, or excipients. Pharmaceutical compositions comprising bioproducts produced using cell lines of the present invention can be further formulated by methods well known in the art (e.g., Remington: The Science and Practice a/Pharmacy, 19th edition (1995), (A. Gennaro et al., Mack Publishing Co.).

### BRIEF DESCRIPTION OF FIGURES

FIG. 1: A graph depicting the temperature-dependent degradation of PS80 mono-oleate ester in the presence of PPT1 over time, which demonstrates that PPT1 degrades PS80 over time in a temperature-dependent manner.

FIG. 2: A graph depicting the degradation of PS80 mono-oleate ester over time in formulated mAb samples: control (A), and spiked separately with (B) LAL—1 ppm, (C) LPL—1 ppm, (D) PPT1—1 ppm, and (E) LPLA2—0.1 ppm, demonstrating that PS80 mono-oleate ester present in the formulation degrades over time to a greater extent in the presence of these proteins than the formulated mAb control.

FIG. 3: A graph depicting the degradation of PS80 mono-oleate ester over time in control sample (A) and in the presence of 0.25 UN/mL PLD4 (B), 2.5 UN/mL PLD4 (D), 0.25 UN/mL PLD7 (C), and 2.5 UN/mL PLD7 (E). This data qualitatively demonstrates the capacity for PLD family members to degrade PS80 over time.

Without limiting the scope of the invention, the following preparations and examples are provided for those of ordinary skill in the art a means of making and using the methods and compositions described herein.

### EXAMPLES

### Example 1—Characterization of Polysorbate Hydrolytic Activity of PPT1

### Polysorbate Degradation Analysis by Liquid Chromatography-Mass Spectrometry (LCMS)—General Procedure A

LCMS analyses are performed on a Waters ACQUITY UPLC (I class) equipped with a Waters SYNAPT® G2-Si mass spectrometer; column: Agilent PLRP-S 2.1×50 mm, 1000 Å, 5 μm particle size; mobile phase: A—0.05% trifluoroacetic acid (TFA) in water, B—0.04% TFA in acetonitrile. Standard solutions are prepared with 2% PS80 and 10 mM citrate buffer to get 0.001, 0.002, 0.005, 0.01, 0.025, 0.05% PS80 solutions. Standard curves of prepared PS80 solutions in 10 mM citrate buffer are obtained in order to quantify intact PS80 in samples by LCMS extracted ion chromatograms for polysorbate mono-oleate. Using the standard curves, the relative percent (%) of intact PS80 as a mono-oleate ester for each sample is calculated against time=zero.

### Example 1a—Polysorbate 80 Degradation in the Presence of PPT1

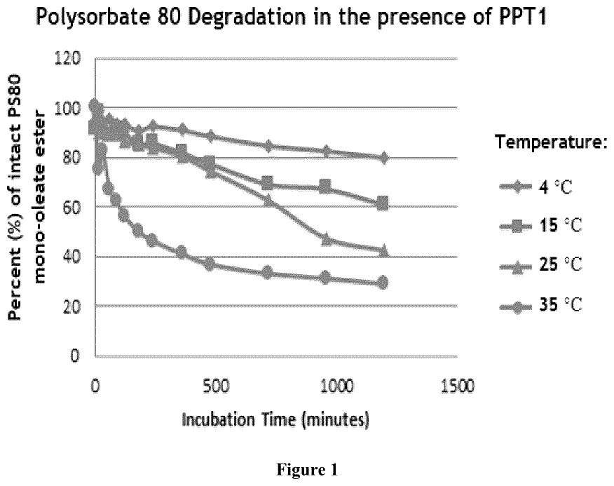

Samples of polysorbate 80 (PS80) and PPT1 are prepared as follows: 0.5 mL of 0.02% w/v PS80 in 10 mM citrate buffer (pH 6) is mixed with 5.6 μL of a 0.3 mg/mL solution of PPT1 (prepared by recombinant expression) and samples are kept at 4, 15, 25, and 35° C. for the duration of the study. Samples (50 μL) of these solutions are taken at time intervals and mixed with 5 μL of 5% formic acid in water for LCMS analysis. The percent of remaining intact PS80 as a mono-oleate ester is monitored by LCMS over time using General Procedure A. These data are shown in FIG. 1 and demonstrate that PPT1 degrades PS80 over time in a temperature-dependent manner.

### Example 1b—Polysorbate 80 Degradation in mAb Formulation Samples Spiked with LAL, LPL, PPT1, and LPLA2

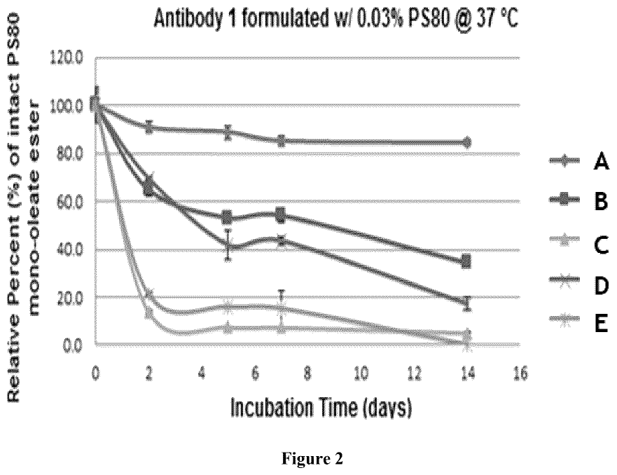

Samples of a formulated mAb (Antibody 1, 100 mg/mL in 20 mM sodium acetate buffer, pH 5.0, with 0.03% w/v PS80) are spiked separately with 1 ppm LAL, LPL, and PPT1, and 0.1 ppm LPLA2 (obtained from recombinant expression). The samples are incubated at 37° C. for the duration of the study. Each sample is diluted with 20 mM sodium acetate buffer in 1:2 ratio and then analyzed by LCMS using General Procedure A. The percent of remaining intact PS80 as a mono-oleate ester over time is shown in Table 1 and FIG. 2.

TABLE 1
Relative Percent (%) vs the Time Zero of Intact PS80 in Samples
of Antibody 1 Spiked with LAL, LPL, PPT1, and LPLA2

| | PS80 mono-oleate ester remaining (average of | | | | |
|---|---|---|---|---|---|
| | relative percent (%) ± standard deviation) after: | | | | |
| | 0 days | 2 days | 5 days | 7 days | 14 days |
| Antibody 1 control | 100 | 90.7 ± 2.3 | 88.7 ± 2.9 | 85.1 ± 2.0 | 84.2 ± 1.3 |
| Antibody 1 spiked | 100 | 64.6 ± 2.2 | 52.9 ± 2.2 | 53.9 ± 2.9 | 34.1 ± 0.1 |
| with LAL, 1 ppm | | | | | |
| Antibody 1 spiked | 100 | 13.9 ± 1.0 | 7.6 ± 0.4 | 7.5 ± 0.5 | 5.2 ± 0.3 |
| with LPL, 1 ppm | | | | | |
| Antibody 1 spiked | 100 | 69.5 ± 0.7 | 41.6 ± 6.2 | 43.5 ± 1.6 | 17.1 ± 2.8 |
| with PPT1, 1 ppm | | | | | |
| Antibody 1 spiked | 100 | 21.1 ± 0.6 | 16.0 ± 0.9 | 15.3 ± 7.7 | 0.2 ± 0.0 |
| with LPLA2, | | | | | |
| 0.1 ppm | | | | | |

Note:
All results in Table 1 represent n = 2

These data demonstrate that PS80 mono-oleate ester present in the formulation degrades over time to a greater extent in the presence of these proteins than the formulated mAb control.

Together the data in this example demonstrate the ability of these proteins (LAL, LPL, PPT1, and LPLA2) to degrade PS80 in solution over time.

### Example 2—Identification of PPT1 in an Fc-Fusion Protein Formulation

Two separate culture batches of an Fc-fusion protein (Fc-Fusion Protein 1) are subjected to Protein A chromatography. Aliquots (25 μL) of the Protein A mainstreams are mixed with of 1M Tris-HCl buffer, pH 8 (5 μL), Barnstead water (172 μL), a protein standard mixture (0.8 μL), and 2.5 mg/mL bovine r-trypsin (2 μL). The samples are incubated at 37° C. for 16 hours. The samples are mixed with 2 μL of a 50 mg/mL dithiothreitol (DTT) solution and then heated at 90° C. for 10 min. The samples are centrifuged at 10,000 g for 2 minutes and the supernatants are transferred into vials. The samples are then acidified with 5% TFA in H₂O (5 μL) and analyzed by LCMS. LCMS analysis is performed on a Waters ACQUITY UPLC equipped with a ThermoFisher Q Exactive™ Plus mass spectrometer; column: Waters UPLC CSH C18, 2.1×50 mm, 1.7 μm particle size; mobile phase: A—0.10% formic acid (FA) in water, B—0.10% FA in acetonitrile, with the column submerged in ice water. In this experiment PPT1 is identified in the samples of Fc-Fusion Protein 1 post-Protein A purification by a non-target proteomics (DDA) approach at 0.5±0.1 ppm (n=2).

### Example 3—Generation of a Recombinantly Engineered LPLA2, LAL, LPL, and PPT1 Knockout CHO Cell Line

Unless otherwise noted, the cell culture media used refers to serum-free cell culture media supplemented with 8 mM glutamine. Additionally, unless otherwise noted, the mammalian cells used are a glutamine synthase deficient CHO (GS-CHO) cell line.

Engineering of cell lines is accomplished through the use of custom-made zinc-finger nuclease (ZFN) reagents designed to be specific for each target HCP gene, constructed by Sigma Aldrich (CompoZr® Custom Zinc Finger Nuclease, Cat. No. CSTZFN, Sigma Aldrich, St. Louis, MO). The ZFN binding/cutting region nucleic acid sequences for LPLA2, LPL, LAL, and PPT1 are given in Table 2.

TABLE 2
ZFN binding/cutting regions for LPLA2,
LPL, LAL, and PPT1.

| SEQ | Gene | ZFN bind/cut nucleic | Bind/ |
|---|---|---|---|
| ID | target | acid sequence (cut sequence | cut |
| NO: | HCP: | lower-case and italicized): | exon: |
| 5 | LPLA2 | TGGATCGCCATCACCTCActtgtcGCG | 1 |
| | | CGACCCAGCTCCGGAG | |
| 6 | LPL | AGCAAAGCCCTGCTCCTGGtggctCTG | 1 |
| | | GGAGTGTGGCTCCAG | |
| 7 | LAL | TACTGGGGATACCCGAGTgaggaGCAT | 2 |
| | | ATGATCCAGAC | |
| 8 | PPT1 | CGCCTTCGCTGACACCGCtggtgATCT | 1 |
| | | GGcATGGGATGGGTA | |

### Preparation of Cells for Gene Disruption—General Procedure B

Vials containing cells are thawed in a 36° C. water bath until only a sliver of ice remains. The cells are seeded into cell culture media in shake-flask culture. The culture of the parental cell line is sub-cultured into cell culture media and is maintained and passaged on a 3-day/4-day schedule. Cell cultures are seeded at a 0.2×10⁶ vc/mL seed density in 30 mL appropriate maintenance medium, as noted above. On the day of transfection, the cells are counted and an appropriate volume of cells is harvested.

### ZFN Transfection and Bulk Culture Recovery—General Procedure C

ZFN transfections are performed using the Nucleofector™ technology and associated cGMP Nucleofector™ Kit V (Cat. No. VGA-1003, Lonza, Basel, Switzerland). Briefly described, enough cells for single Nucleofection reactions (2-4.5×10⁶ vc) are collected by centrifugation. Following complete removal of the supernatant, the cell pellet is suspended in 100 μL of Nucleofector™ solution V, with supplement added, according the manufacturer's protocol. The suspended cells are gently mixed by trituration and transferred to a vial containing an aliquot of the ZFN mRNA [part of the custom ZFN kit generated by Sigma Aldrich (St. Louis, MO)]. The cell/mRNA mixture is then transferred to a 2 mm cuvette provided in the Nucleofector™ kit, the cuvette is inserted into the Nucleofector™ device, and the cells are electroporated. Following electroporation, the cells rest at room temperature in the cuvette for 30-60 seconds, and then they are transferred using a sterile transfer pipet to a well in a labeled 6-well plate (Falcon Cat. no. 351146, Corning, Durham, NC) containing 3 mL cell culture media. The transfected cells are maintained in the 6-well plate, static, in a humidified incubator for 1-4 days at 36° C., 6% CO₂, after which they are transferred to shake-flask culture in cell culture media, 36° C., 6% CO₂, shaking 125 rpm, until the viability is >90%. Once the cells are recovered completely from transfection (as measured by viability in shake-flask culture), the bulk culture is single-cell sorted using FACS technology.

The ZFN transfections for each target HCP may be performed a single time prior to single-cell sorting. Alternatively, the ZFN transfections for any particular target HCP may performed two times, with complete cell recovery prior to the second ZFN transfection. More than one round of ZFN transfection may increase the number of cells containing a bi-allelic mutation in the respective target HCP, making screening more efficient.

### Detection of ZFN-Mediated Target HCP Sequence Modifications in Bulk Cultures—General Procedure D

Two to seven days post-transfection, cells from the partially-to-fully recovered ZFN bulk cultures are harvested for evaluation to assess the activity of the transfected ZFN. The Surveyor® Mutation Detection Assay (MDA) (Transgenomic Inc., Omaha, NE) is used to detect the efficiency of the ZFN procedure in generating modifications at the target HCP site, according to the manufacturer's protocol. Briefly, the ZFN-binding region is PCR amplified using primers provided in the CompoZr® Custom Zinc Finger Nuclease kit (Sigma, St. Louis, MO). The PCR products are then denatured and re-annealed. The Cel-I endonuclease (Surveyor Nuclease S) provided in the MDA kit is used to detect DNA mismatch “bubbles”, derived from the annealing of PCR products consisting of the native or wild-type sequence and those that contain indels, as Cel-I will recognize these “bubbles” of mismatch and cleave the DNA. After the Cel-I digest, products are then resolved on a 2% or 4% TBE agarose gel (Reliant Gel, Lonza, Basel, Switzerland). In the absence of DNA mismatch “bubbles”, no DNA cleavage will occur and only one band will be present, representing the PCR product. If any non-homologous end-joining (NHEJ) occurred, representing ZFN activity, cleavage products will be observed on the gel in the form of two (or more) bands. Only those ZFN bulk cultures that show a positive response in the MDA are forward-processed to single-cell sorting.

### Single-Cell Sorting by Fluorescence-Activated Cell Sorting—General Procedure E

The recovered bulk culture is sorted via Fluorescence-Activated Cell Sorting (FACS) technology. The protocols and methods for the single-cell cloning are well-known in the art. For cloning, a cell sorter (MoFlo™ XDP, Beckman Coulter) is used to identify and sort single, viable cells by measuring laser diffraction in the forward and side-scatter directions, according to methods which are well-known in the art (see, for example, Krebs, L., et al. (2015) “Statistical verification that one round of fluorescence-activated cell sorting (FACS) can effectively generate a clonally-derived cell line.” BioProcess J 13(4): 6-19).

Cells are sorted into 96-well microtiter plates (Falcon, catalog number 35-3075) containing animal-component free sort medium (Ex-Cell CHO cloning media, SAFC C6366)+20% conditioned cell culture medium+phenol red (Sigma P0290)). To prepare conditioned cell culture medium, parental cells are seeded at a density of 1×10⁶ vc/mL into a cell culture medium without glutamine and incubated in a shake-flask at 36° C., 6% CO₂, 125 rpm for 20-24 h. The culture is centrifuged to remove cells and the conditioned media is filtered through a sterile 0.22 μm filter. Seven to ten days post single-cell sort, all the plates are fed with 50 μL cell culture media per well. On day 14-15 post single-cell sort, the plates are analyzed for clonal outgrowth. Outgrowth is determined by imaging of the sort plates using a CloneSelect Imager (Molecular Devices, Sunnyvale, CA) or manually with the aid of a mirror and/or by observation of a medium color change from red to orange/yellow.

### Screening Clonally-Derived Cell Lines for ZFN-Mediated Target HCP Sequence Modifications—General Procedure F

Clonally-derived cell lines (CDCLs) are picked from 96-well plates that originate from the recovered ZFN bulk culture as they become a visible colony and are transferred to deep 96-well plates (Greiner, Catalog No. 780271) containing cell culture medium. Clonally-derived cell lines are consolidated into deep-well plates containing 150 μL cell culture medium. The cultures are maintained in cell culture medium under static conditions on a 3-day/4-day feed/pass schedule until screening and characterization is complete.

Clonally-derived cell lines (CDCLs) are screened for indels using the Surveyor® MDA. Genomic DNA is isolated from each cell line using the Promega Wizard® SV 96 Genomic DNA Purification Kit (cat. no. A2371, Promega, Madison, WI), according to the manufacturer's protocol. The ZFN PCR reactions are performed using the Phusion© High-Fidelity DNA Polymerase (New England BioLabs, Ipswich, MA), according to manufacturer's protocol. MDA digestion products are resolved on 2% TBE agarose gels. Cell lines which have been identified that are positive in the MDA are characterized through either General Procedure G or General Procedure H.

### Characterizing Indels in CDCLs Using RT-PCR—General Procedure G:

CDCLs are characterized by sequencing of the ZFN PCR products using a target gene RT-PCR reaction. Total RNA is isolated from each potential KO cell line using the RNeasy Micro Kit (Qiagen, Cat. No. 74004, Germantown, MD), according to manufacturer's protocol. Reverse transcription reactions are done using the SuperScript™ III First-Strand Synthesis System for RT-PCR (cat. no. 18080-051, Invitogen, Carlsbad, CA), according to manufacturer's protocol, followed by PCR reactions using the Phusion® High-Fidelity DNA Polymerase (New England BioLabs, Ipswich, MA), according to manufacturer's protocol. The RT-PCR products are resolved on 1% TAE agarose gels, identifying cell lines with altered RT-PCR products. The cell line chosen for forward-processing lacks a RT-PCR product and does not contain the target HCP protein by LCMS.

Characterizing Indels in CDCLs using Next-Generation Sequencing (NGS)—General Procedure H:

MDA-positive CDCLs are consolidated into 96-well deep-well plates for further maintenance. When consolidating, those cell lines that show “off-normal” PCR and/or MDA results are characterized using next-generation sequencing (NGS) provided by GENEWIZ. Cell lines containing acceptable bi-allelic indels in the target HCP gene locus are evaluated by LCMS, carrying forward a cell line which does not contain the target HCP protein.

### Scaling and Banking Knockout Cell Lines—General Procedure I:

Those CDCLs that, based on the initial screen/characterization work, warrant further evaluation are scaled from the 96-well deep-well plates (DWPs) to shake-flasks, and research cell banks (RCB) are generated. From the DWP, cells from the appropriate wells are transferred to an appropriately labeled well in a 6-well plate containing 3 mL cell culture medium. The scaling CDCLs are maintained in the 6-well plate, static, in a humidified incubator for 3 to 4 days at 36° C., 6% CO₂, after which they are transferred to shake-flasks, containing 15 mL of cell culture medium, 36° C., 6% CO₂, shaking at 125 rpm. The shake-flask cultures are passed at least one time to build suitable cell mass for banking. For each cell line, a 3-10 vial RCB is generated with 10-13×10⁶ vc per vial in Freezing Menstrum (90:10 cell culture medium:DMSO). The vials are placed in a styrofoam rack “sandwich” at −80° C. for at least 24 h to allow for a controlled-rate freezing of the cells. Once the vials are completely frozen they are stored at −80° C.

### Example 3a—LPLA2 Knockout CHO Cell Line

CHO cells are prepared for gene disruption according to General Procedure B. The cells are then subjected to a single ZFN transfection and bulk culture recovery according to General Procedure C. Using General Procedure D, sequence modifications in bulk culture are detected. Bulk cultures showing a positive response in the MDA are forward-processed to single-cell sorting according to General Procedure E. Clonally-derived cell lines obtained therefrom are screened for target HCP sequence modifications according to General Procedure F. Indels are characterized according General Procedure G, and a cell line is chosen which does not contain detectable amounts of the LPLA2 protein by LCMS. An RCB is generated according to General Procedure I to give an LPLA2 knockout CHO cell line.

### Example 3b—LPLA2/LPL Knockout CHO Cell Line

LPLA2 knockout CHO cells from Example 3a are prepared for gene disruption according to General Procedure B. The cells are then subjected to two ZFN transfections and bulk culture recovery according to General Procedure C. Using General Procedure D, sequence modifications in bulk culture are detected. Bulk cultures showing a positive response in the MDA are forward-processed to single-cell sorting according to General Procedure E. Clonally-derived cell lines obtained therefrom are screened for target HCP sequence modifications according to General Procedure F. Indels are characterized according General Procedure H and a cell line is chosen which does not contain detectable amounts of the LPL protein by LCMS. An RCB is generated according to General Procedure I to give an LPLA2/LPL knockout CHO cell line.

### Example 3c—LPLA2/LPL/LAL Knockout CHO Cell Line

LPLA2/LPL knockout CHO cells from Example 3b are prepared for gene disruption according to General Procedure B. The cells are then subjected to two ZFN transfections and bulk culture recovery according to General Procedure C. Using General Procedure D, sequence modifications in bulk culture are detected. Bulk cultures showing a positive response in the MDA are forward-processed to single-cell sorting according to General Procedure E. Clonally-derived cell lines obtained therefrom are screened for target HCP sequence modifications according to General Procedure F. Indels are characterized according to General Procedure H and a cell line is chosen which does not contain detectable amounts of the LAL protein by LCMS. An RCB is generated according to General Procedure I to give an LPLA2/LPL/LAL knockout CHO cell line.

### Example 3d—LPLA2/LPL/LAL/PPT1 Knockout CHO Cell Line

LPLA2/LPL/LAL knockout CHO cells from Example 3c are prepared for gene disruption according to General Procedure B. The cells are then subjected to two ZFN transfections and bulk culture recovery according to General Procedure C. Using General Procedure D, sequence modifications in bulk culture are detected. Bulk cultures showing a positive response in the MDA are forward-processed to single-cell sorting according to General Procedure E. Clonally-derived cell lines obtained therefrom are screened for target HCP sequence modifications according to General Procedure F. Indels are characterized according General Procedure H, however none of the cell lines contain bi-allelic mutations in the targeted PPT1 region. Cell lines containing mono- or bi-allelic indels are evaluated by LCMS, carrying forward a cell line which does not contain detectable amounts of the PPT1 protein by LCMS evaluation. An RCB is generated according to General Procedure I to give an LPLA2/LPL/LAL/PPT1 knockout CHO cell line.

### Example 4—Comparison of Polysorbate Stability in Formulated mAbs Expressed in a LPLA2/LPL/LAL/PPT1 Knockout CHO Cell Line Vs. Control

An Fc-fusion protein (Fc-Fusion Protein 1) and an antibody (Antibody 2) are produced from product expressing CHO cell lines with LPLA2, LPL, LAL, and PPT1 knocked out (referred to as “lipase/esterase KO cell line”) and also product expressing CHO cell lines without LPLA2, LPL, LAL, or PPT1 knockouts as a control. Fc-Fusion Protein 1 is processed through Protein A chromatography, low pH viral inactivation, anion exchange chromatography (AEX), cation exchange (CEX) chromatography, and tangential flow filtration (TFF) concentration prior to formulation with 0.02% PS80. Antibody 2 is processed through Protein A chromatography, low pH viral inactivation, CEX chromatography, and TFF concentration prior to formulation with 0.02% PS80. Formulated samples of Fc-Fusion Protein 1 and Antibody 2 are kept at 25° C. for the duration of the study and used directly for LCMS analysis, using General Procedure A to monitor the percent of remaining intact PS80 as a mono-oleate ester over time. The results are listed in Table 3 and indicate that PS80 in Fc-Fusion Protein 1 and Antibody 2 produced using the KO cell line are more stable than the control samples.

TABLE 3
Relative Percent (%) vs the Time Zero of Intact PS80
in Samples of Antibody 2 and Fc-Fusion Protein 1

| | PS80 mono-oleate ester remaining (average of | | | |
|---|---|---|---|---|
| | relative percent (%) ± standard deviation) after: | | | |
| | 0 weeks at | 2 weeks at | 4 weeks at | 8 weeks at |
| Sample: | 25 °C | 25 °C | 25 °C | 25 °C |
| Antibody 2 from | 100 | 83 ± 3 | 77 ± 2 | 69 ± 1 |
| lipase/esterase KO | | | | |
| cell line | | | | |
| Antibody 2 control | 100 | 35 ± 8 | 24 ± 5 | 15 ± 4 |
| Fc-Fusion Protein 1 | 100 | 104 ± 5 | 113 ± 6 | 93 ± 4 |
| from lipase/esterase | | | | |
| KO cell line | | | | |
| Fc-Fusion Protein 1 | 100 | 68 ± 4 | 54 ± 4 | 33 ± 7 |
| control | | | | |

Note:
All results represent n = 3

### Example 5—Identification of PLD3 in a Monoclonal Antibody Formulation

Samples containing 1 mg of Antibody 3 which have been processed through Protein A capture, low pH viral inactivation, anion exchange (AEX) chromatography, and concentration by tangential flow filtration (TFF) to a concentration of 150 mg/mL are mixed with Tris-HCl buffer (1 M, pH 8, 5 μL) and water to achieve a volume of 195 μL. Each solution is treated with 5 μL of tryspin and protein standard mixture (20 μL of 2.5 mg/mL r-bovine trypsin, 20 μL of a protein standard mixture and 60 μL of water) at 37° C. overnight. Each sample is mixed with 1,4-dithiothreitol (DTT, 50 mg/mL, 2 μL) and heated to 90° C. for 10 min, observing a white precipitate. The samples are then centrifuged at 13000 g for 2 min and the supernatant is transferred into a HPLC vial. The samples are acidified with 5 μL of 10% formic acid in water before LCMS analysis essentially as described for Example 2. In this experiment PLD3 is identified in the samples of Antibody 3 at 17±6 ng/mg (n=2) of Antibody 3.

### Example 6—Characterization of Polysorbate Hydrolytic Activity of PLD4 and PLD7

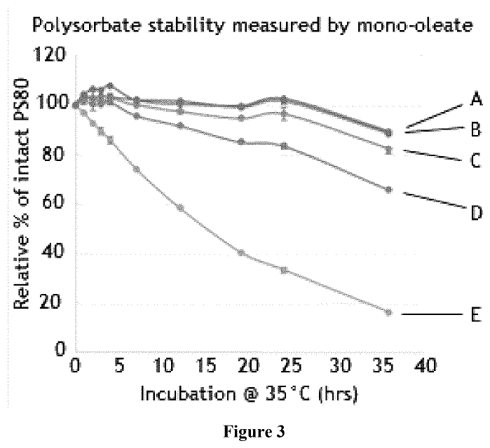

PLD4 and PLD7, like PLD3, are phospholipase D family members. The hydrolytic activity of PLD4 and PLD7 is assessed in a manner that is essentially as described in Example 1. Samples containing 0.02% PS80 are incubated with 0.25 and 2.5 units per milliliter (UN/mL) of PLD4 and PLD7 at 35° C., and the percent of remaining intact PS80 as a mono-oleate ester is monitored by LCMS over time using General Procedure A. After 35 h incubation under these conditions, PS80 is >30% and >80% hydrolyzed in the presence of 2.5 UN/mL PLD4 and PLD7, respectively. These data are shown in FIG. 3, and qualitatively demonstrate the capacity for PLD family members to degrade PS80 over time.

1.-39. (canceled)

40. A recombinantly engineered mammalian cell having reduced expression and/or reduced activity of host cell proteins (HCPs) palmitoyl-protein thioesterase 1 (PPT1) and the endogenous HCPs lipoprotein lipase (LPL), lysosomal acid lipase (LAL), and phospholipase A2 (LPLA2) comprising a disrupted or inactivated gene encoding the PPT1, the LAL, the LPL, and the LPLA2, wherein said cell further comprises a polynucleotide encoding dulaglutide.

41. The cell of claim 40, further having reduced expression and/or reduced activity of host cell protein phospholipase D3 (PLD3) and comprising a disrupted or inactivated gene encoding PLD3.

42. The cell of claim 40, wherein the cell produces a protein A-binding fraction having substantially reduced polysorbate degradation activity relative to the polysorbate degradation activity of a cell without the disruptions or inactivation of the genes.

43. The cell of claim 42, wherein the reduction in degradation of intact polysorbate is greater than 30%.

44. The cell of claim 43, wherein the reduction in degradation of intact polysorbate is greater than 50%.

45. The cell of claim 40, wherein the cell is a CHO cell.

46. The cell of claim 45, wherein the cell is a CHO-K1 cell, a CHOK1SV cell, a DG44 CHO cell, a DUXB11 CHO cell, a CHO-S, a CHO GS (glutamine synthetase) knock-out cell, a CHOK1SV FUT8 knock-out cell, a CHOZN, or a CHO-derived cell.

47. A method of producing dulaglutide comprising the steps of:
(a) obtaining a sample comprising dulaglutide and a plurality of host cell proteins from a host cell modified to produce reduced or eliminated levels of PPT1 compared to an unmodified cell; and
(b) subjecting the sample to at least one purification step to remove at least one host cell protein, wherein the host cell comprises:
i) a modification to reduce or eliminate the expression in a coding sequence of a polynucleotide encoding a PPT1 protein; and
ii) modifications to reduce or eliminate the expression in coding sequences of polynucleotides encoding fatty acid hydrolases of LAL, LPL, and LPLA2.

48. The method of claim 47, wherein the plurality of host cell proteins (a) does not comprise a detectable amount of a PPT1 protein; and (b) does not comprise a detectable amount of at least one other lipase or esterase.

49. The method of claim 47, wherein the purification step is protein A affinity (PA) chromatography or another affinity chromatography method, cation exchange (CEX) chromatography, anion exchange (AEX) chromatography or hydrophobic interaction chromatography (HIC).

50. A process for reducing polysorbate degradation in a dulaglutide formulation comprising the steps of:
(a) modifying a host cell to reduce or eliminate the expression of PPT1 protein;
(b) modifying the host cell to reduce or eliminate the expression of LAL, LPL, and LPLA2;
(c) transfecting the cell with a polynucleotide encoding dulaglutide;
(d) extracting a protein fraction comprising dulaglutide from the host cell;
(e) contacting the protein fraction with a chromatography media which is PA chromatography or another affinity chromatography method, CEX chromatography, AEX chromatography or HIC;
(f) collecting dulaglutide from the media;
(g) combining dulaglutide with a fatty acid ester; and
(h) optionally, adding a buffer; or
(i) optionally, adding one or more pharmaceutically acceptable carriers, diluents, or excipients.

51. A process for reducing aggregation or particle formation in a dulaglutide formulation comprising the steps of:
(a) modifying a host cell to reduce or eliminate the expression of PPT1 protein;
(b) modifying the host cell to reduce or eliminate the expression of LAL, LPL, and LPLA2;
(c) transfecting the cell with a polynucleotide encoding dulaglutide;
(d) extracting a protein fraction comprising dulaglutide from the host cell;
(e) contacting the protein fraction with a chromatography media which is PA chromatography or another affinity chromatography method, CEX chromatography, AEX chromatography or HIC; and
(f) collecting dulaglutide from the media; and
(g) combining dulaglutide with a fatty acid ester; and
(h) optionally, adding a buffer; or
(i) optionally, adding one or more pharmaceutically acceptable carriers, diluents, or excipients.

52. A process for producing a stable formulated dulaglutide comprising the steps of:
(a) modifying a host cell to reduce or eliminate the expression of PPT1 protein;
(b) modifying the host cell to reduce or eliminate the expression of LAL, LPL, and LPLA2;
(c) transfecting the cell with a polynucleotide encoding dulaglutide;
(d) extracting a protein fraction comprising dulaglutide from the host cell;
(e) contacting the protein fraction with a chromatography media which is PA chromatography or another affinity chromatography method, CEX chromatography, AEX chromatography or HIC;
(f) collecting dulaglutide from the media;
(g) combining dulaglutide with a fatty acid ester; and
(h) optionally, adding a buffer; or
(i) optionally, adding one or more pharmaceutically acceptable carriers, diluents, or excipients.

53. The process of claim 50, wherein the (a) of modifying the host cell to reduce or eliminate the expression of PPT1 comprises inserting or deleting at least one nucleotide within exon 2, exon 3 or exon 4 of a polynucleotide encoding the PPT1 protein.

54. The process of claim 51, wherein the (a) of modifying the host cell to reduce or eliminate the expression of PPT1 comprises inserting or deleting at least one nucleotide within exon 2, exon 3 or exon 4 of a polynucleotide encoding the PPT1 protein.

55. The process of claim 52, wherein the (a) of modifying the host cell to reduce or eliminate the expression of PPT1 comprises inserting or deleting at least one nucleotide within exon 2, exon 3 or exon 4 of a polynucleotide encoding the PPT1 protein.

56. The process of claim 53, wherein the polynucleotide encoding the PPT1 protein comprises a nucleic acid sequence that is at least 80% identical to SEQ ID NO:1.

57. The process of claim 54, wherein the polynucleotide encoding the PPT1 protein comprises a nucleic acid sequence that is at least 80% identical to SEQ ID NO:1.

58. The process of claim 55, wherein the polynucleotide encoding the PPT1 protein comprises a nucleic acid sequence that is at least 80% identical to SEQ ID NO:1.


SEQUENCE LISTING (19 sequences)

SEQ ID NO: 1 - protein, 306 aa, Cricetulus griseus
MASPGSRWLLAVSLLPWCCAAWSLGHLNPPSLTPLVIWHGMGDSCCNPISMGAIKKMVEKEIPGIYVLSLEIGKNMMEDVENSFFLNVNSQVMMVCQILEKDPKLQQGYNAIGFSQGGQFLRAVAQRCPSPRMINLISVGGQHQGVFGLPRCPGESSHVCDFIRKMINAGAYSKVVQLRLVQAQYWHDPIKEDVYRNHSIFLADINQERCVNETYKKNLMALNKFVMVKFLNDSIVDPVDSEWFGFYRSGQAKETIPLQESTLYTEDRLGLKQMDKAGKLVFLAKEGDHLQLSKEWFNAYIIPFLK

SEQ ID NO: 2 - protein, 397 aa, Cricetulus griseus
MQILGLVVCLFLSVLLSGRPTGSIPHVDPEANMNVTEMIRYWGYPSEEHMIQTEDGYILGVHRIPHGRKNHSHKGPKPVVYLQHGFLADSSNWVTNSDNSSLGFILADAGFDVWLGNSRGNTWSLKHRTLSISQDEFWAFSFDEMAKYDLPASIYYIVNKTGQEQVYYVGHSQGTTIGFIAFSQIPELAKKIKMFFALAPVVFLNFALSPVIKISKWPEVIIEDLFGHKQFFPQSAKLKWLSTHVCNRVVLKKLCTNVFFLICGFNEKNLNESRVNVYTSHSPAGTSVQNLRHWGQIAKHHMFQAFDWGSKAKNYFHYNQTCPPVYDLKDMLVPTALWSGDHDWLADPSDVNILLTQIPNLVYHKRLPDWEHLDFLWGLDAPWRMYNEIVNLLRKYQ

SEQ ID NO: 3 - protein, 473 aa, Cricetulus griseus
  VARIANT 24: note = X can be any naturally occurring amino acid
MESKALLLVALGVWLQSLTASQGXAAADGGRDFTDIESKFALRTPDDTAEDNCHLIPGIAESVSNCHFNHSSKTFVVIHGWTVTGMYESWVPKLVAALYKREPDSNVIVVDWLYRAQQHYPVSAGYTKLVGNDVARFINWMEEEFNYPLDNVHLLGYSLGAHAAGVAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCNSKEAFEKGLCLSCRKNRCNNVGYEINKVRAKRSSKMYLKTRSQMPYKVFHYQVKIHFSGTESDKQLNQAFEISLYGTVAESENIPFTLPEVSTNKTYSFLIYTEVDIGELLMMKLKWKSDSYFSWSDWWSSPGFVIEKIRVKAGETQKKVIFCAREKVSHLQKGKDSAVFVKCHDKSLKKSG

SEQ ID NO: 4 - protein, 412 aa, Cricetulus griseus
MDRHHLTCRATQLRSGLLVPLLLLMMLADLALSVQRHPPVVLVPGDLGNQLEAKLDKPKVVHYLCSKRTDSYFTLWLNLELLLPVIIDCWIDNIRLVYNRTSRATQFPDGVDVRVPGFGETFSLEFLDPSKRTVGSYFHTMVESLVGWGYTRGEDLRGAPYDWRRAPNENGPYFLALREMIEEMYQMYGGPVVLVAHSMGNMYTLYFLQRQPQAWKDKYIHAFISLGAPWGGVAKTLRVLASGDNNRIPVIGPLKIREQQRSAVSTSWLLPYNHTWSHDKVFVHTPTTNYTLRDYHQFFQDIRFEDGWFMRQDTEGLVEAMMPPGVELHCLYGTGVPTPDSFYYESFPDRDPKICFGDGDGTVNLESVLQCQAWQSRQEHKVSLQELPGSEHIEMLANATTLAYLKRVLFEP

SEQ ID NO: 5 - DNA, 43 bp, Cricetulus griseus
  source 1..43: mol_type = genomic DNA
tggatcgccatcacctcacttgtcgcgcgacccagctccggag

SEQ ID NO: 6 - DNA, 42 bp, Cricetulus griseus
  source 1..42: mol_type = genomic DNA
agcaaagccctgctcctggtggctctgggagtgtggctccag

SEQ ID NO: 7 - DNA, 38 bp, Cricetulus griseus
  source 1..38: mol_type = genomic DNA
tactggggatacccgagtgaggagcatatgatccagac

SEQ ID NO: 8 - DNA, 42 bp, Cricetulus griseus
  source 1..42: mol_type = genomic DNA
cgccttcgctgacaccgctggtgatctggcatgggatgggta

SEQ ID NO: 9 - protein, 488 aa, Cricetulus griseus
MKPKLMYQELKVPVEEPAGELPVNEIEAWKAAEKKARWVLLVLILAVVGFGALMTQLFLWEYGDLHLFGPNQRPAPCYDPCEAVLVESIPEGLEFPNATTSNPSTSQAWLGLLAGAHSSLDIASFYWTLTNNDTHTQEPSAQQGEEILQQLQALAPRGVKVRIAVSKPNGPLADLQSLLQSGAQVRMVDMQKLTHGVLHTKFWVVDQTHFYLGSANMDWRSLTQVKELGVVMYNCSCLARDLTKIFEAYWFLGQAGSSIPSTWPRPFDTRYNQETPMEICLNGTPALAYLASAPPPLCPSGRTPDLKALLSVVDSARSFIYIAVMNYLPTMEFSHPRRFWPAIDDGLRRAAYERGVKVRLLVSCWGHSEPSMRSFLLSLAALRDNHTHSDIQVKLFVVPADEAQARIPYARVNHNKYMVTERAVYIGTSNWSGSYFTETAGTSLLVTQNGHDGLRSQLEDVFLRDWNSLYSHNLDTAADSVGNACRLL

SEQ ID NO: 10 - DNA, 39 bp, Cricetulus griseus
  source 1..39: mol_type = genomic DNA
gccccctgctatgacccctgcgagtaagtggcaggggag

SEQ ID NO: 11 - protein, 575 aa, Cricetulus griseus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNDLGKDHEILRRRIENGAKELWFFLQSELKKLKKLEGNELQRHADEILLDLGHHERSIMTDLYYLSQTDGAGEWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVKDKNVQVVELPIVDSLHPRPPYLPLAVPEDLADRLLRVHGDPAVWWVSQFVKYLIRPQPWLEREIEETTKKLGFKHPVIGVHVRRTDKVGTEAAFHPIEEYMVHVEEHFQLLERRMKVDKKRVYLATDDPSLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHQPRTKEEIPMEPGDIIGVAGNHWNGYSKGVNRKLGKTGLYPSYKVREKIETVKYPTYPEAEK

SEQ ID NO: 12 - protein, 408 aa, Cricetulus griseus
MQTLGILLLAVGLLAASASAVIRIPLRKFTSIRRTMTEVGGSVEDLILKGPITKYSNQSPAETKGPVSELLKNYLDAQYYGEIGIGTPPQCFTVVFDTGSSNLWVPSIHCKLLDIACWIHHKYNSGKSSTFVKNGTSFDIHYGSGSLSGYLSQDTVSVPCKSEQPGGLKVEKQIFGEAIKQPGITFIAAKFDGILGMGYPSISVNNVVPVFDNLMQQKLVEKNIFSFFLNRDPTGQPGGELMLGGIDSKYYEGELSYLNVTRKAYWQVHMDQLDVANGLTLCKGGCEAIVDTGTSLLVGPVDEVKELQKAIGAVPLIQGEYMIPCEKVSSLPSVTLKLGGKDYELSPSKYVLKVSQGGKTICLSGFMGMDIPPPSGPLWILGDVFIGTYYTVFDRDNNRVGFAKAATL

SEQ ID NO: 13 - DNA, 41 bp, Cricetulus griseus
  source 1..41: mol_type = genomic DNA
cagtgtcagagttgctcaaaaactacctggatgtgagtgat

SEQ ID NO: 14 - protein, 1255 aa, Cricetulus griseus
  VARIANT 83: note = X can be any naturally occurring amino acid
AGPLLPGRPQVKLVGNMHGDETVSRQVLVYLAHELASGYRRGDPRLVRLLNITDVYLLPSLNPDGFERSREGDCGLGDSGSPXAPPRRGRDLNRSFPDQFSTGKPPSLDEVPEVRALIDWIRKNKFVLSGNLHGGSVVASYPFDDSPDHMATGIYSKTSDDEVFRYLAKAYASNHPIMKTGEPHCPGDEDETFKDGITNGAHWYDVEGGMQDYNYVWANCFEITLELSCCKYPPASQLRQEWENNRESLITLIEKVHIGIKGFVKDSVTGAGLENATISVAGINHNITTGRFGDFHRLLIPGIYNLTAVSTGYMPLTIHNIRVKEGPATEMDFSLRPTVTSKVPDSTEAVATPGTVAVPNIPPGTSSSHQPIQPKDFHHHHFPDMEIFLRRFANEYPNITRLYSLGKSVESRELYVMEISDNPGVHEPGEPEFKYIGNMHGNEVVGRELLLNLIEYLCKNFGTDPEVTDLVRSTRIHLMPSMNPDGYEKSQEGDSVSVVGRNNSNNFDLNRNFPDQFVTITDPTQPETIAVMSWIKSYPFVLSANLHGGSLVVNYPFDDNEQGVATYSKSPDDAVFQQIALSYSRENSQMFQGRPCKDMSILNEYFLHGITNGASWYNVPGGMQDWNYLQTNCFEVTIELGCVKYPFEKELPKYWEQNRRSLIQFMKQVHQGVKGFVLDATDGRGILNATLSVAEINHPVTTYKAGDYWRLLVPGTYKITASARGYNPVTKNVTVRSEGAIQVNFTLVRSSTDANNESKKGKGASTSTDDSSDPTTKEFEALIKHLSAENGLEGFMLSSSSDLALYRYHSYKDLSEFLRGLVMNYPHITNLTTLGQSAEYRHIWSLEISNKPNVSEPEEPKIRFVAGIHGNAPVGTELLLALAEFLCLNYKKNPVVTQLVDRTRIVIVPSLNPDGRERAQEKECTSKIGQTNARGKDLDTDFTSNASQPETKAIIENLIQKQDFSLSIALDGGSVLVTYPYDKPVQTVENKETLKHLASLYANNHPSMHMGQPSCPNKSDENIPGGVMRGAEWHSHLGSMKDYSVTYGHCPEITVYTSCCYFPSAAQLPALWAENKRSLLSMLVEVHKGVHGLVKDKTGKPISKAVIVLNDGIKVHTKEGGYFHVLLAPGVHNINAIAEGYQQQHSQVFVHHDAASSVLIVFDTDNRIFGLPRELVVTVSGATMSALILTACIIWCICSIKSNRHKDGFHRLRQHHDEYEDEIRMMSTGSKKSLLSHEFQDETDTEEETLYSSKH

SEQ ID NO: 15 - DNA, 39 bp, Cricetulus griseus
  source 1..39: mol_type = genomic DNA
gtcagtggagtcaagagaactgtatgtgatggagatatc

SEQ ID NO: 16 - protein, 585 aa, Cricetulus griseus
MAAPMDRSPGGRAVRALRLALALASLTEVLLNCPAGALPTQGPGRRRQNLDPPVSRVRSVLLDAASGQLRLVDGIHPYAVAWANLTNAIRETGWAYLDLGTNGSYNDSLQAYAAGVVEASVSEELIYMHWMNTMVNYCGPFEYEVGYCEKLKSFLEINLEWMQREMELSQDSPYWHQVRLTLLQLKGLEDSYEGRLTFPTGRFTIKPLGFLLLQIAGDLEDLEQALNKTSTKLSLGSGSCSAIIKLLPGARDLLVAHNTWNSYQNMLRIIKKYQLQFRQGPQEAYPLIAGNNLVFSSYPGTIFSGDDFYILGSGLVTLETTIGNKNPALWKYVQPQGCVLEWIRNIVANRLALDGATWADIFKQFNSGTYNNQWMIVDYKAFIPNGPSPGSRVLTILEQIPGMVVVADKTEDLYKTTYWASYNIPFFEIVFNASGLQDLVAQYGDWFSYTKNPRAQIFQRDQSLVEDMNSMVRLIRYNNFLHDPLSLCEACIPKPNAENAISARSDLNPANGSYPFQALYQRPHGGIDVKVTSFSLAKRMSMLAASGPTWDQLPPFQWSLSPFRSMLHMGQPDLWTFSPISVPWD

SEQ ID NO: 17 - DNA, 42 bp, Cricetulus griseus
  source 1..42: mol_type = genomic DNA
cggttcctgctccgctatcatcaagttgctgccaggcgcacg

SEQ ID NO: 18 - protein, 199 aa, Cricetulus griseus
MSSGNAKIGYPAPNFKATAVMPDGQFRDICLSEYRGKYVVFFFYPLDFTFVCPTEIIAFSDRAEEFKKLNCQVIGASVDSHFCHLAWINTPKKQGGLGPMNIPLVSDPKRTIAQDYGVLKADEGISFRGLFIIDDKGILRQITINDLPVGRSVDEILRLVQAFQFTDKHGEVCPAGWKPGSDTIKPDVQKSKEYFSKQK

SEQ ID NO: 19 - DNA, 43 bp, Cricetulus griseus
  source 1..43: mol_type = genomic DNA
cctgcccccaacttcaaagccacagctgttatgccagatggac